(19) United States
(12) Patent Application Publication
(10) Pub. No.: US 2008/0167823 A1
(43) Pub. Date: Jul. 10, 2008

(54) IMPEDANCE SPECTROSCOPY (IS) METHODS AND SYSTEMS FOR CHARACTERIZING FUEL

(75) Inventors: Charles Koehler, Milwaukee, WI (US); Martin Seitz, Brookfield, WI (US); Richard Hirthe, Milwaukee, WI (US); David Wooton, Beaverdam, VA (US)

Correspondence Address: WHYTE HIRSCHBOECK DUDEK S.C., 33 East Main Street, Suite 300, Madison, WI, 53703-4655, US

(73) Assignee: Paradigm Sensors, LLC, Milwaukee, WI (US)

(21) Appl. No.: 11/963,311

(22) Filed: Dec. 21, 2007

Related U.S. Application Data
(60) Provisional application No. 60/871,690, filed on Dec. 22, 2006.
(60) Provisional application No. 60/871,694, filed on Dec. 22, 2006.

(51) Int. Cl. G01N 33/22; G06F 19/00

(52) U.S. Cl. 702/23; 702/65

(57) ABSTRACT

The present invention relates to methods and systems or apparatuses for analyzing fluids. More particularly the present invention relates to apparatuses and methods that employ impedance spectroscopy (IS) for analyzing fuels. Fuels of interest include biofuel, particularly biodiesel. Hand-held and “in-line” IS apparatuses are disclosed.

### CROSS REFERENCE TO RELATED APPLICATIONS

Priority is claimed of U.S. Provisional patent application Ser. Nos. 60/871,694 and 60/871,690 both filed on Dec. 22, 2006.

### FIELD OF THE INVENTION

The present invention relates to impedance spectroscopy or impedance spectroscopic methods and systems or apparatuses for characterizing or analyzing fluids. More particularly the present invention relates to apparatuses and methods that employ impedance spectroscopy (IS) for analyzing fuels. Fuels of interest include biofuel, particularly biodiesel. Yet more specifically this invention relates to portable, preferably hand-held, IS apparatuses systems and methods.

### BACKGROUND OF THE INVENTION

Increasing consumption of fossil fuels is occurring on a worldwide basis. Many countries rely on fossil fuel use to the detriment of society and ecosystems. Reduction in the amount of fossil fuel consumption and increased use of bio-based fuels has become an increasingly important initiative for consumers and governments alike. In particular, the increased use of biodiesel is lauded as an important step in the direction of reducing fossil fuel consumption and usage. However, the transition to biodiesel in everyday fuel has created a series of problems to both diesel consumers and combustion engine manufacturers. A key problem surrounds determining the concentration of biofuel, often equated with or referred to as fatty acid methyl ester (FAME), concentration or volume percentage of a biodiesel sample. Identification of other alkyl esters is contemplated by this invention.

Biodiesel is often defined as the monoalkyl esters of fatty acids from vegetable oils and animal fats. Neat and blended with conventional petroleum diesel fuel, biodiesel has seen significant use as an alternative diesel fuel. Biodiesel is often obtained from the neat vegetable oil transesterification with an alcohol, usually methanol (other short carbon atom chain alcohols may be used), in the presence if a catalyst, often a base. Various unwanted materials are found in biodiesel, which can include glycerol, residual alcohol, moisture, unreacted feedstock (triacylglycerides), monoglycerides, diglycerides, and free (unreacted) fatty acids.

Biodiesel fuels are often blended compositions of diesel fuel and biomass, which is often esterified soy-bean oils, rapeseed oils or various other vegetable oils. It is the similar physical and combustible properties to diesel fuel that has allowed the development of biofuels as an energy source for combustion engines. However, biofuels are not a perfect replacement for diesel. By example, the cetane number, oxidation stability and corrosion potential of these biofuels present a concern to continued consumption as a viable fuel. Based upon these issues, as well as others known to one skilled in the art, careful control of the biofuel concentration must be implemented.

Beyond the physical and chemical concerns, monetary concerns exist. The United States government provides a tax credit for biofuel consumption. The tax credit is based upon the biofuel percentage within a biodiesel blend. In fact, the tax credit can be substantially different for a slight change in the percentage, since $0.01 per FAME percentage per gallon used is provided by the government. Therefore the difference between 20% and 25% FAME (volume percent is used throughout) in biodiesel fuel can result in a considerable tax value. Often it is the case that biodiesel blends are “splash-blended”, which refers to the liquid agitation that occurs as the fuel truck is driving on the road after the diesel and biofuel have been combined. “Splash-blended” biodiesel blends often have a blend variance of up to 5%, which is unacceptable.

Various methods and technologies have been employed to determine the biofuel percentage within a biodiesel blend. These methods include gas chromatography (GC), fourier transform infrared (FTIR) spectroscopy, and near-infrared (NIR) spectroscopy. None of these methods provide a portable, quick and accurate determination of the fatty acid alkyl (FAAE) e.g., FAME percentage within a biodiesel blend.

It would be advantageous to have a system and method for quickly and accurately determining the concentration of biodiesel fuel blends for use in quality control, production testing and distribution testing. This invention provides the basis upon which IS can be used to characterize fuel, particularly biofuel, in a convenient, cost-effective and timely manner.

### BRIEF SUMMARY OF THE INVENTION

Briefly, the present invention involves impedance spectroscopy or impedance spectroscopic (IS) methods and systems or apparatuses for characterizing fuel. In one aspect the present invention is methods for characterizing fuel using IS data, In a further aspect, the present invention is apparatuses or systems for obtaining and analyzing IS data to characterize fuel, usually a relatively discrete sample thereof. The kind of fuel characterized by use of this invention is biofuel (discussed in more detail below), particularly biodiesel. The particular characteristic of biofuel which is a primary focus of this invention is that of biomass percentage which is also discussed in detail below. Many other physical or chemical characteristics of fuel, and combinations and subcombination of such characteristics, can be analyzed by use of this invention. A hand-held or easily portable IS apparatus is one preferred system of this invention. In-line, (as in a fuel processing plant, a fuel supply line or fuel storage structure such as a fuel tank (fixed or on a vehicle), or other real-time sampling), discrete sampling, continuous sampling, and all other approaches to obtain IS data from fuel are herein contemplated. One skilled in this art, in light of the disclosure of this invention, will appreciate that IS methods, systems, or apparatuses can be used to characterize many chemical and physical qualities of fuel. One skilled in this art will also appreciate, in light of this disclosure, that system size, components thereof, their interrelationship(s), configuration, sampling technique, parameter measurement, and data treatment, storage, retrieval and display can all be adapted to obtain desired fuel characterization information.

It is to be understood that “fuel” as that term is used herein is intended to mean any material that is capable of being characterized using IS technology and which is or can be used to initiate and sustain combustion. Liquid fuels capable of being analyzed using IS technology are a recognized class of fuels that are a focus of this invention. Note that this definition of fuel includes materials whose states can be changed at elevated or reduced (i.e., from ambient) temperature or pressure to permit IS data collection. Liquefied natural gas (LNG), liquefied alkanes, e.g., propane, are fuels within the contemplation of this invention. One skilled in this art will appreciate that the sampling technique and conditions and sample cell/probe design employed to obtain IS data may be adapted to the fuel being analyzed.

### BRIEF DESCRIPTION OF THE DRAWINGS

FIG. 1 is a block diagram of the fuel analyzer system in accordance with at least one embodiment of the invention.

FIG. 2 is a block diagram of a logic controller in accordance with at least one embodiment of the invention.

FIG. 3 is an alternative embodiment of the fuel analyzer system in accordance with at least one embodiment of the invention.

FIG. 4 is a flow chart representing a method for analyzing biodiesel blends in accordance with at least one embodiment of the invention.

FIG. 5 is a FTIR spectra for biodiesel concentration.

FIG. 6 is a Beer's Law FTIR model for biodiesel concentration standards.

FIG. 7 is a room temperature impedance spectra for biodiesel standards.

FIG. 8 is an impedance spectroscopy model for biodiesel concentration standards.

FIG. 9 is a test data table including both FTIR and impedance spectroscopy data.

FIG. 10 is a biodiesel method comparison data plot.

FIG. 11 is a biodiesel method residuals data plot.

FIG. 12 is an alternative embodiment of the impedance spectroscopy data analyzer in accordance with at least one embodiment of the present invention.

FIG. 13 is a measured form calculation sequence.

FIG. 14 is a complex Plane Representation mathematical sequence.

FIG. 15 is an impedance and modulus plot sequence.

FIG. 16 is a biodiesel modulus spectra plot.

FIG. 17 is an impedance spectroscopy derived model data plot.

### DETAILED DESCRIPTION OF THE PREFERRED EMBODIMENT

Biodiesel includes fuels comprised of short chain, mono-alkyl, preferably methyl, esters of long chain fatty acids derived from e.g., vegetable oils or animal fats. Short carbon atom chain alkyl esters have from e.g., 1 to 6 carbon atoms, preferably 1 to 4 carbon atoms and most preferably 1 to 3 carbon atoms. Biodiesel is also identified as B100, the “100” representing that 100% of the content is biodiesel. Biodiesel blends include a combination of both petroleum-based diesel fuel and biodiesel fuel. Typical biodiesel blends include B5 and B20, which are 5% and 20% biodiesel respectively. Diesel fuel is often defined as a middle petroleum distillate fuel.

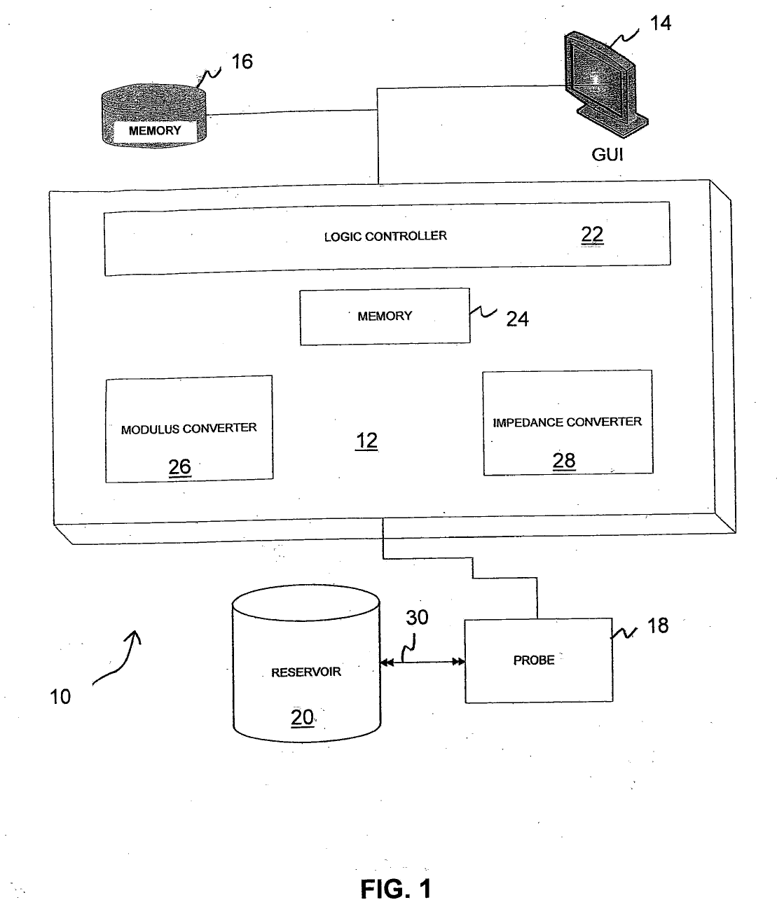

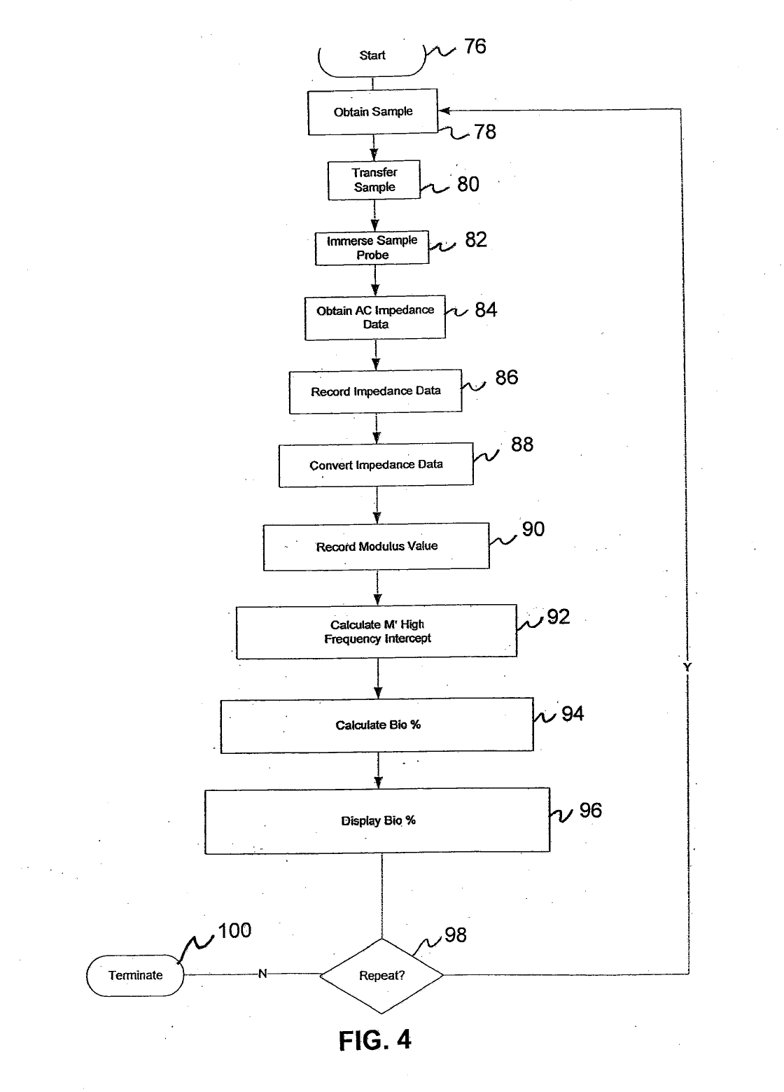

Now referring to FIG. 1, an illustrative example of the system 10 in accordance with at least one embodiment of the invention includes an analysis device 12, graphical user interface (GUI) 14, memory storage device 16, probe 18, and reservoir 20. The analysis device 12 includes a logic controller 22, a memory storage device 24, a modulus converter 26 and an impedance converter 28. The reservoir 20 contains a biofuel sample, which can be selected from the group including a biodiesel blend, heating fuel, second phase materials, fuel additives, methanol, glycerol, residual alcohol, moisture, unreacted feedstock (triacylglycerides), monoglycerides, diglycerides, and free (unreacted) fatty acids. The probe 18 is external and separately connected to the reservoir 20 and can alternatively be integrated within the reservoir 20. Probe 18 (or more generally probe means, sampling apparatus or means, sampling cell or sample cell, as appropriate) may be a discrete separate structure or it may be part of an assembly, e.g., a sample cell. It is to be understood that probe as used herein means essentially any apparatus of the appropriate size and configuration which can be used to gather IS data from a fuel sample. Probe 18 provides inputs to the reservoir 20 through input/output line 30. Excitation voltage (V_{(f)}) is applied to the reservoir from probe 18 and a response current (I_{(f)}) over a range of frequencies is measured and provided to the analyzer 12. The impedance data is analyzed and converted by the impedance converter 28, and then transferred to the modulus converter 28. The impedance data includes Zᵣₑₐₗ, Z_{imaginary}, and frequency. The modulus data includes Mᵣₑₐₗ, M_{imaginary}, and frequency. The logic controller 22 operates the modulus converter 26 and impedance converter 28 to store the respective data, including the impedance measurements, within memory 24. The logic controller performs a computer readable function, which is accessed from memory 24, that performs an impedance spectroscopy analysis method (See FIG. 4) and provides a biodiesel concentration to the GUI 14. The concentration data can be provided in the form of Bxx, where “xx” represents the concentration of the sample tested that is biofuel (biomass/FAME) in percentage of biodiesel. Concentration and percentage are often used interchangeably to describe the amount of biodiesel within a blended sample.

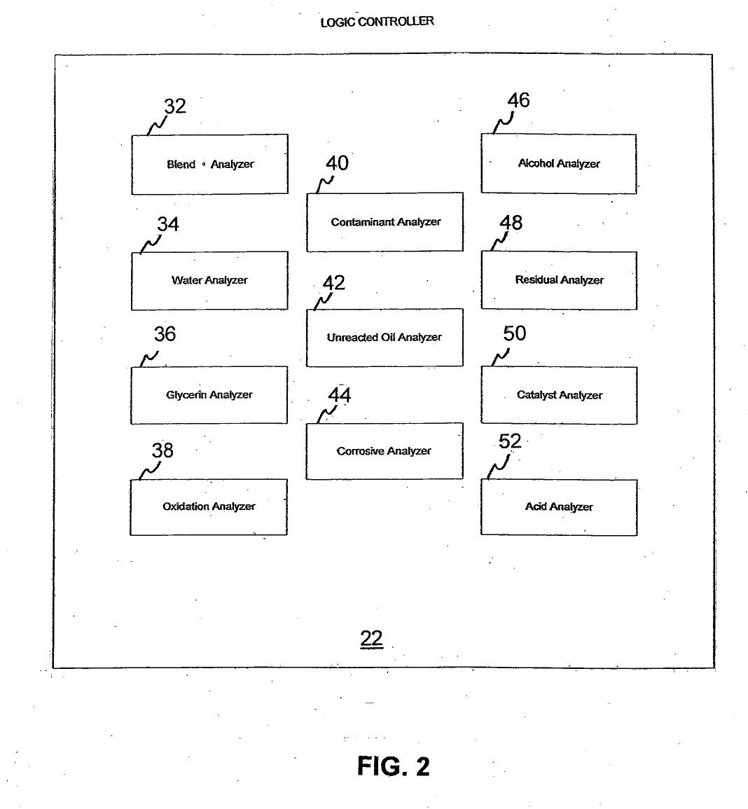

Referring to FIG. 2, an alternative embodiment of the logic controller 22 is illustrated. The controller 22 includes a blend concentration analyzer 32, a water analyzer 34, a glycerin analyzer 36 (generally total glycerine meaning the sum of bound and free glycerine or glycenol), an oxidation analyzer 38, a contaminant analyzer 40, and unreacted oil analyzer 42, a corrosive analyzer 44, an alcohol analyzer 46, a residual process chemistry analyzer 48, a catalyst analyzer 50, and a total acid number (e.g., fatty acid or carboxylic acid) analyzer 52. The water analyzer 34 performs analysis on the impedance data obtained from probe 18 cf., A.S.T.M. D6584 or D6751. (Acid number and alcohol/methanol analysis are generally of greater interest regarding B100, i.e., neat biodiesel.) The controller 22 accesses a computer readable function accessed from memory 24 and provides information such as the presence of water, and if identified within the sample, the concentration of water within the sample. The glycerin analyzer 36 performs analysis on the impedance data obtained from probe 18. The controller 22 accesses a computer readable function accessed from memory 24 and provides information such as the presence of glycerin, and if identified within the sample, the concentration of glycerin within the sample. Alternatively, the computer readable function is accessed from memory 16. In an alternative embodiment, a viscosity analyzer (not shown), and cetane number analyzer (not shown) are included for providing viscosity data and cetane number data for a fuel sample. In yet another alternative embodiment, a sludge/wax analyzer (not shown) are included for providing information on the presence and amount of sludge and/or wax precipitation within a fuel sample.

The oxidation analyzer 38 performs analysis on the impedance data obtained from probe 18. The controller 22 accesses a computer readable function accessed from memory 24 and provides information such as the presence of oxidation. The contaminant analyzer 40 performs analysis on the impedance data obtained from probe 18. The controller 22 accesses a computer readable function accessed from memory 24 and provides information such as the presence of contaminants, and identification of the type of contaminants within the sample, as well as the concentration of the particular contaminant within the sample. A variety of contaminants can be found within fuel samples, which include water, wax/sludge, and residual process chemistry.

The unreacted oil analyzer 42 performs analysis on the impedance data obtained from probe 18. The controller 22 accesses a computer readable function from memory 24 and provides information such as the presence of unreacted oils, as well as the concentration within the sample. A variety of unreacted oil can be found within fuel samples, which include unreacted feedstock (triacylglycerides), monoglycerides, diglycerides, and free (unreacted) fatty acids or carboxylic acids.

The corrosive analyzer 44 performs analysis on the impedance data obtained from probe 18. The controller 22 accesses a computer readable function from memory 24 and provides information such as the presence of corrosives, as well as the reactivity of the corrosive substances within the sample.

The alcohol analyzer 46 performs analysis (e.g., for methanol) on the impedance data obtained from probe 18. The controller 22 accesses a computer readable function from memory 24 and provides information such as the presence of alcohol, and if present, the concentration of alcohol within the sample. The residual analyzer 48 performs analysis on the impedance data obtained from probe 18. The controller 22 accesses a computer readable function memory 24 and provides information such as the presence of residuals, and identification of the type of residuals within the sample, as well as the concentration of the residuals within the sample. A variety of residuals can be found within fuel samples, which include alcohol, catalyst, glycerin and unreacted oil.

The catalyst analyzer 50 performs analysis on the impedance data obtained from probe 18. The controller 22 accesses a computer readable function from memory 24 and provides information such as the presence of catalysts, as well as the concentration of the catalysts within the sample. A variety of catalysts can be found within fuel samples, which include KOH and NaOH. The total acid number analyzer 52 performs analysis on the impedance data obtained from probe 18. The controller 22 accesses a computer readable function from memory 24 and provides information such as the presence of acids, as well as the concentration of the acids within the sample. A variety of acids can be found within fuel samples, which include carboxylic acid and sulfuric acid.

In an alternative embodiment, a stability analyzer (not shown) is provided. The stability analyzer performs analysis on the impedance data obtained from probe 18. The controller 22 accesses a computer readable function accessed from memory 24 and provides information such as a stability value. Recent research has found that changes to the biodiesel element of biodiesel blends can have a deleterious effect upon the stability of the fuel sample over time. Blended samples that are left inactive for extended periods of time can potentially lose stability. The impedance spectroscopy data and stability analyzer function of this invention can provide information as to the sample's stability and efficacy.

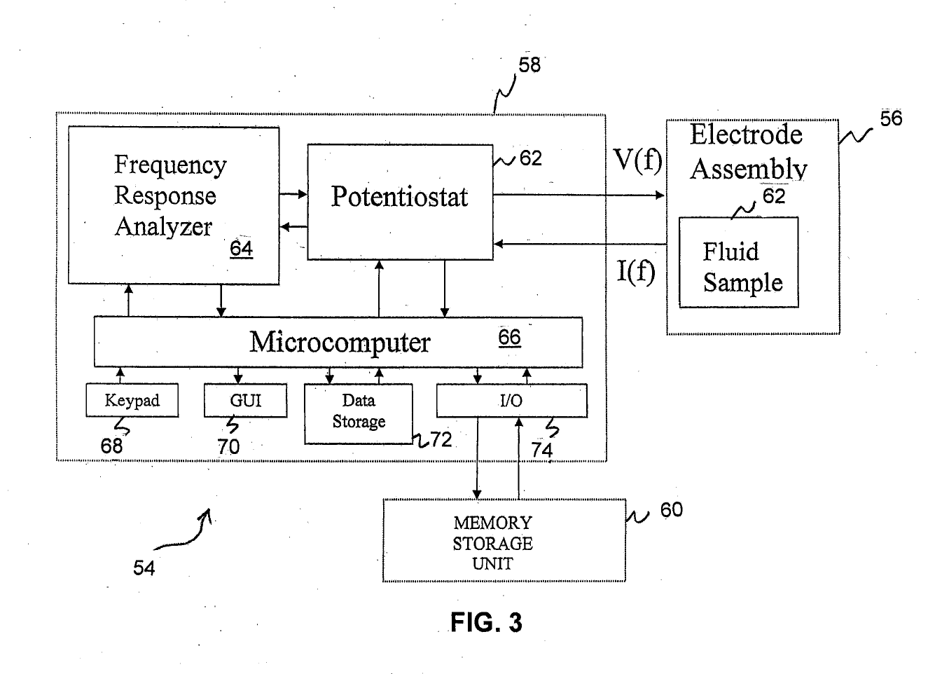

Referring to FIG. 3, an alternative embodiment of the impedance spectroscopy analyzing system 54 is provided. The system 54 includes an electrode assembly 56 a data analyzer 58, and a memory storage unit 60. The electrode assembly 56 includes a fluid sample 62 and probes (not shown). The data analyzer 58 includes a potentiostat 62, a frequency response analyzer 64, a microcomputer 66, a keypad 68, a GUI (graphical user interface) 70, data storage device 72, and I/O device 74. Impedance data is obtained from the electrode assembly 56 and input into the analyzer 58. The potentiostat 62 and frequency response analyzer together perform the impedance spectroscopy analysis methods (See FIG. 4). The microcomputer 66 accesses the computer readable functions from the data storage device 60 or 72, and provide biofuel analyzed data to the GUI 70

Referring to FIG. 4, a flow chart is provided representing a method for determining the concentration of biodiesel (e.g., biomass/FAME content) in a blended biodiesel fuel sample in accordance with at least one embodiment of the present invention. The system 10 is initiated at step 76. A sample of the blended biodiesel is obtained at step 78 and then transferred to a clean container or reservoir at step 80. The sample is maintained at substantially room temperature, generally between about 60° F. and about 85° F. Alternatively, the sample is located in a vehicle fuel tank on board a vehicle or deployed “in-line” e.g., in a biodiesel synthesis plant. Measurement probes are cleaned and immersed within the reservoir at step 82. Alternatively, probes can be maintained within the reservoir and the fuel sample is added to the reservoir with the probes already within the reservoir. The probes can be self-cleaning probes. The impedance device is initiated and the AC impedance characteristics of the fuel sample are obtained at step 84. The frequency range extends from about 10 milliHertz to about 100 kHertz, or alternatively appropriate frequencies. The impedance data is recorded at step 86. The data can be saved in a memory device integral to the device 12. Alternatively, the impedance data is saved in an external memory device. The external memory device 16 can be a relational database or a computer memory module. At step 88, the impedance data is converted to complex modulus values. The complex modulus values are recorded at step 90. M′ high frequency intercept values are determined at step 92 from the complex modulus values and the biodiesel concentration is calculated at step 94. By example, Equation Set 1 is a linear algorithm used for calculating the biodiesel blend concentration. The biodiesel concentration value is represented on a user interface at step 96. If the process continues, steps 78 through 98 are repeated, otherwise the sequence is terminated at step 100. One skilled in the art would recognize that there are many chemical and physical differences between biodiesel and petroleum-based diesel which the present invention can characterize.

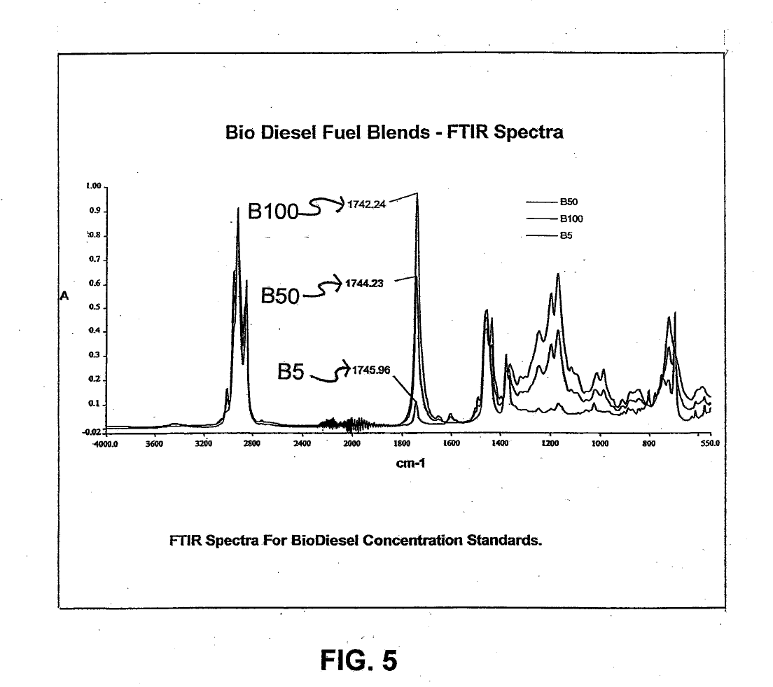

The Fourier transform infrared (FTIR) spectra analysis of three concentration biodiesel samples is provided in FIG. 5. Samples of B100, B50, and B5 were tested using an FTIR process. The FTIR process used for data obtained in FIG. 5 was modeled after the AFNOR NF EN 14078 (July 2004) method, titled “Liquid petroleum products—Determination of fatty acid methyl esters (FAME) in middle distillates—Infrared spectroscopy method.” Biodiesel fuel samples were diluted in cyclohexane to a final analysis concentration of about 0% to about 1.14% biofuel. This was to produce a carbonyl peak intensity that ranged between about 0.1 to about 1.1 Abs, using a 0.5 mm cell pathlength. The method showed a 44 g/l sample (B5 sample was diluted to 0.5%) having 0.5 Abs carbonyl peak height. The method recommended 5-standards be prepared ranging from about 1 g/l (about 0.11% biofuel) to about 10 g/l (about 1.14% biofuel).

The peak height of the carbonyl peak at or near 1245 cm⁻¹ was measured to a baseline drawn between about 1820 cm⁻¹ to about 1670 cm⁻¹. This peak height was used with a Beer's Law plot of absorbance versus concentration to develop a calibration curve for unknown calculation.

The modifications made to this method included no sample dilution, an alternated total reflectance (ATR) cell and utilization of peak area calculations. Sample dilution with cyclohexane is a very large source of errors. The reasons to dilute the sample include reducing the viscosity for flow (transmission cell), opacity or to maintain the absorption peak height of the sample with the detector linearity. The detector linearity of the instrument used was in the range of about 0 Abs to about 2.0 Abs. By reducing the cell pathlength to about 0.018 mm the absorbance of a B100 sample was about 1.0 Abs. This allowed dilution to be unnecessary. The use of a UATR cell allowed a very controlled and fixed pathlength to be maintained.

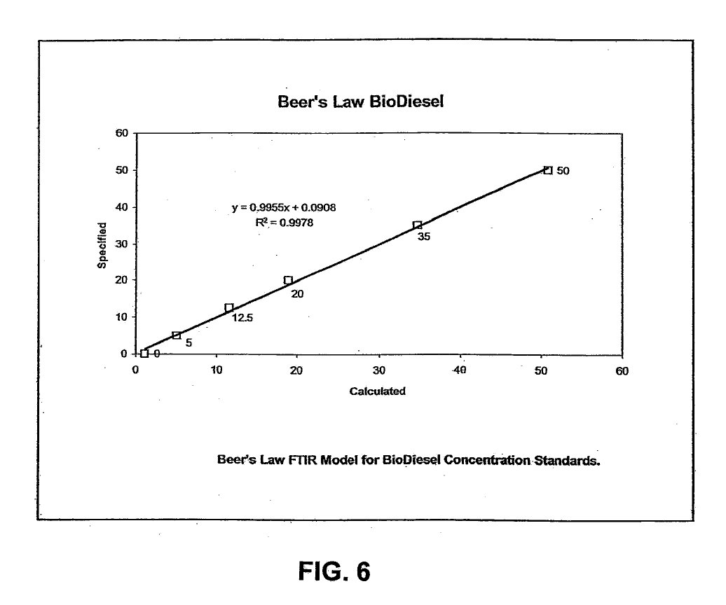

The peak of interest demonstrated migration during dilution due to solvent interaction, evidenced in the biofuel spectra shown in FIG. 5. As a result, the peak area was chosen as the measurement technique. In addition, peak area is the preferred technique for samples that contain multiple types of a defined chemistry type, such as that found in biofuels. Substances found in biofuels that are distinguishable from one another and from petroleum-based fuels constituents by means of impedance spectroscopy are, of course, a focus of this invention. Exemplary substances include saturated and unsaturated esters. The result of Beer's Law calibration is shown in FIG. 6. The biofuel samples were measured against the calibration curve of FIG. 6. The impedance spectroscopy methods were measured against this FTIR process.

y=−3.371E+07x+8.158E+09, Equation Set 1

where y=M′ and x=% biodiesel

At least one embodiment of the present invention was tested for feasibility by comparison with FTIR analysis, an industry accepted test method, of biodiesel fuel blend concentration. The blend samples that were tested included B50, B20 and B5. The samples were evaluated using both broad spectrum AC impedance spectroscopy as well as FTIR spectroscopy. Additionally, the blends of unknown values were tested to determine the impedance data using impedance spectroscopy. Conventional diesel fuel and a variety of nominal blend ratios were used as test standards.

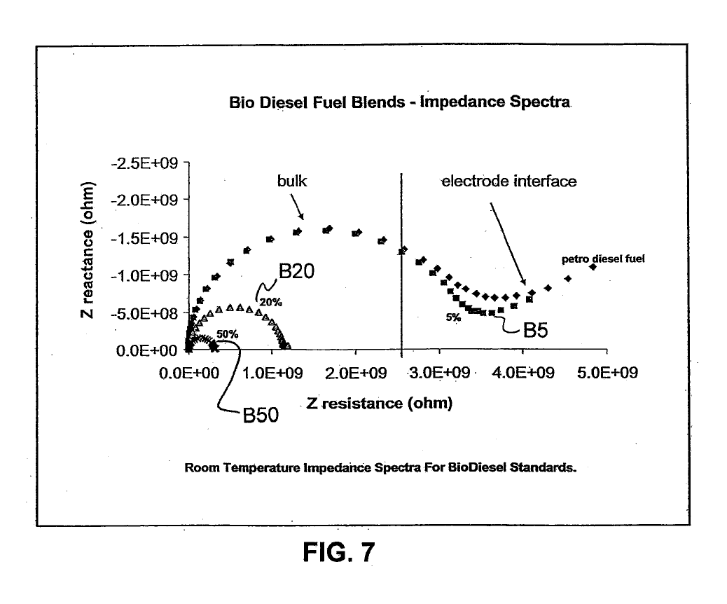

Approximately 20 mL samples of each biodiesel blend were evaluated at room temperature utilizing a two (2) probe measurement configuration. FIG. 5 provides an example of the impedance spectra in a line plot configuration, with reactance (ohm) plotted against resistance (ohm). The impedance spectra provide a clear distinction between B50, B20, B5, and petroleum diesel fuel. Generally the impedance at given frequency, ω, contains two contributions as shown in Equation Set 2. More specifically, FIG. 7 provides the resistance (Rₛ) plotted against the Reactance (1/ωCₛ), which provides an indication that the resistivity of the biodiesel blend sample is sensitive to the percent biodiesel within the base diesel fuel. As a result, the impedance spectra can be used to identify the concentration percentage of biodiesel within a biodiesel blend sample.

Z*(ω)=Rₛ−j(1/ωCₛ) Equation Set 2

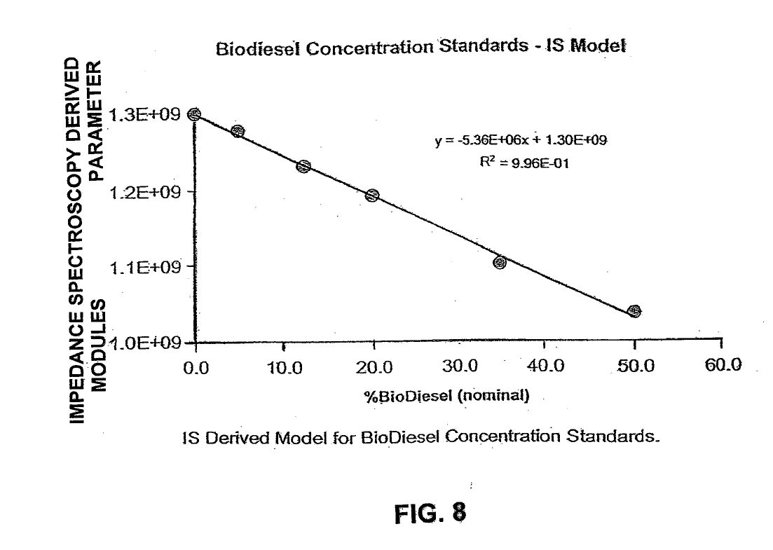

Further manipulation of the impedance data indicates that the polarizability of the blended biodiesel sample is systematically impacted as the concentration of biodiesel increases or decreases. Therefore, a real modulus representation value can be calculated. This presents a parameter, for which a correlation can be made. A correlation between the measured impedance-derived spectra data and the stated biodiesel percentage concentration value can be established. The correlation is graphically presented in FIG. 8, where the impedance derived modulus parameter is plotted against the biodiesel concentration. A linear relationship having a negative slope is provided. These results provide an indication that a correlation similar to that of the industry accepted FTIR method is feasible for impedance spectroscopy.

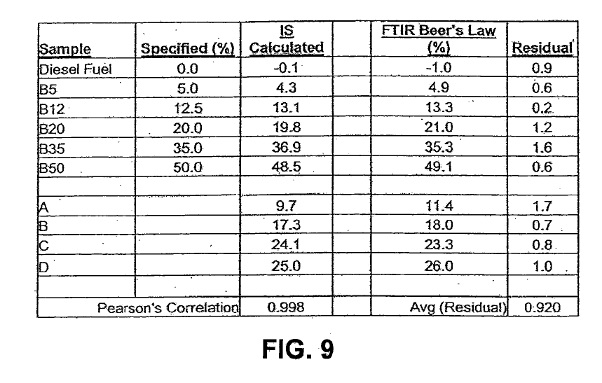

Referring to FIG. 9, a test data table is provided. The table includes known biodiesel standards, including pure petroleum diesel fuel, B5, B12, B20, B35, and B50. Each of these standards (Reference Standards) was tested using the FTIR process and the impedance spectroscopy process of the present embodiment. The results for each of these tests are provided in the table. Additionally there are four unknowns, A, B, C, and D (Unknown Blend Set 1), for which test results were obtained using both the FTIR process and the impedance spectroscopy process of the present embodiment.

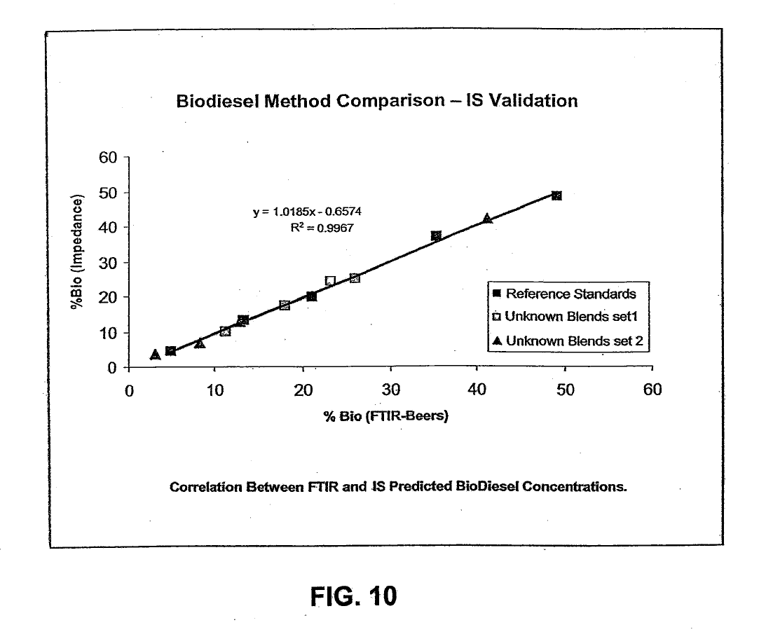

Referring to FIG. 10, the test data provided in FIG. 9 is presented in the form of an X-Y plot. The biodiesel concentration data obtained from the impedance spectroscopy process is plotted against the biodiesel concentration data obtained from the FTIR process. A correlation line is fit to the data points, which indicates a close correlation between the two methods for determining biodiesel concentration. Additionally, a second set of unknown biodiesel blends (Unknown Blends Set 2) were tested through both stated processes. These unknown blends were prepared by blending B100 and two separate petroleum fuels. These data points are not provided in FIG. 9, but are plotted in FIG. 10.

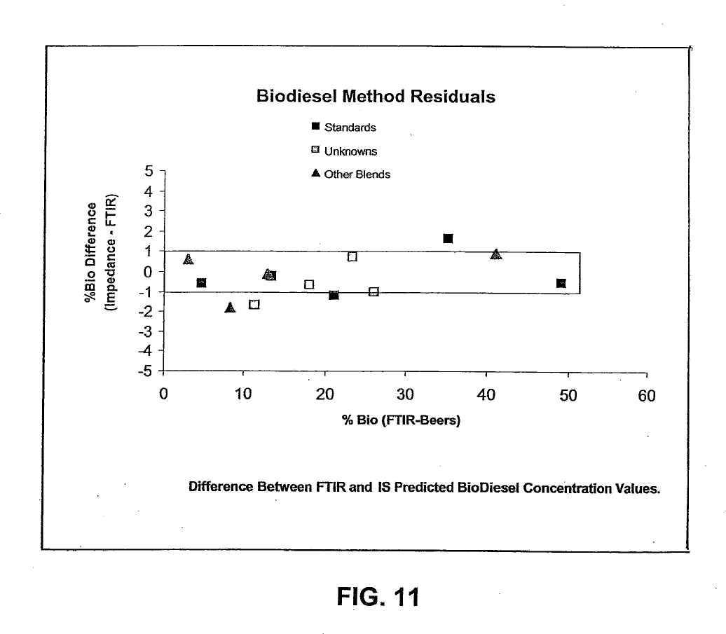

A scientifically significant agreement between the FTIR process and the impedance spectroscopy process of the present embodiment was found. This is evidenced by the line fit assigned to the plotted data points. Residual values (% biO_{FTIR}−% bio_{Impedance}) were calculated and provided in FIG. 9. The average residual value is 0.920, which is less than 1.0%, presenting a highly significant linear correlation between the widely accepted FTIR process and the impedance spectroscopy process of the present embodiment. The difference between the FTIR process and the impedance spectroscopy process of the present embodiment are presented in FIG. 11.

The system 10 is implemented in the form of a low cost, portable device for determining real-time evaluation of biodiesel blends. The device provides the user with blended FAME concentration in order for the user to compare with established specifications. Furthermore, the device enables the user to detect contaminants and unwanted materials within the biodiesel sample. The impedance spectroscopy data processing provides the user a broader functionality view of the biodiesel sample, and not simply the chemical make-up. Performance of the fuel can be affected by unwanted materials and detecting the presence of the unwanted materials the user is better able to make decisions that affect performance of the vehicle.

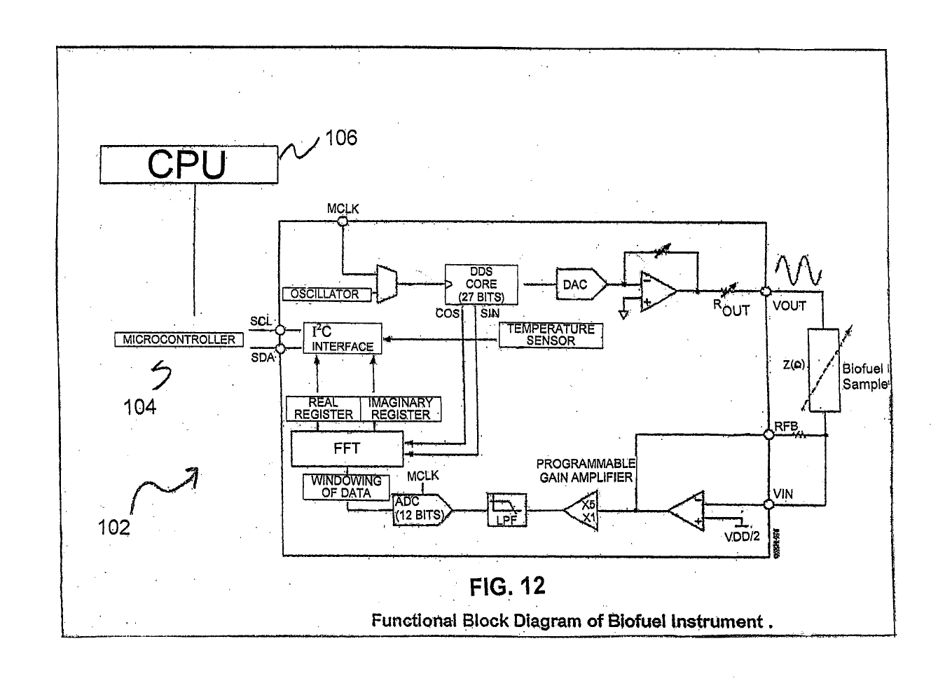

An alternative embodiment of the impedance spectroscopy system 102 is shown in FIG. 12. The biofuel sample is tested external to the system 102, or alternatively internal (not shown) to the system 102. A microcontroller 104 relays data to the central processing unit (CPU) 106 for calculation. Once the data has been calculated the biofuel concentration is sent to a graphical user interface (GUI) (not shown) by an I/O device (not shown). The present embodiment is a portable bench-top device 102. The device 102 has either an internal or external power source and a suitable sampling fixture. The impedance data is acquired by the device 102 and transferred to the CPU for detection and identification, of elements within the sample as well as the relative concentrations of the elements. By example, the elements can include FAAE (fatty acid alkyl esters), FAME, glycerol, residual alcohol, moisture, additives, corrosive compounds, unreacted feedstock (triacylglycerides), monoglycerides, diglycerides, and free (unreacted) fatty acids.

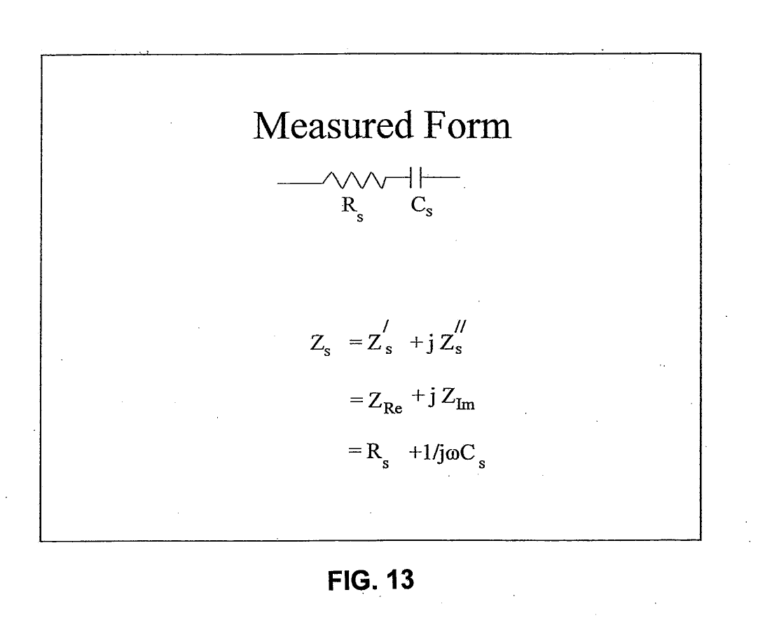

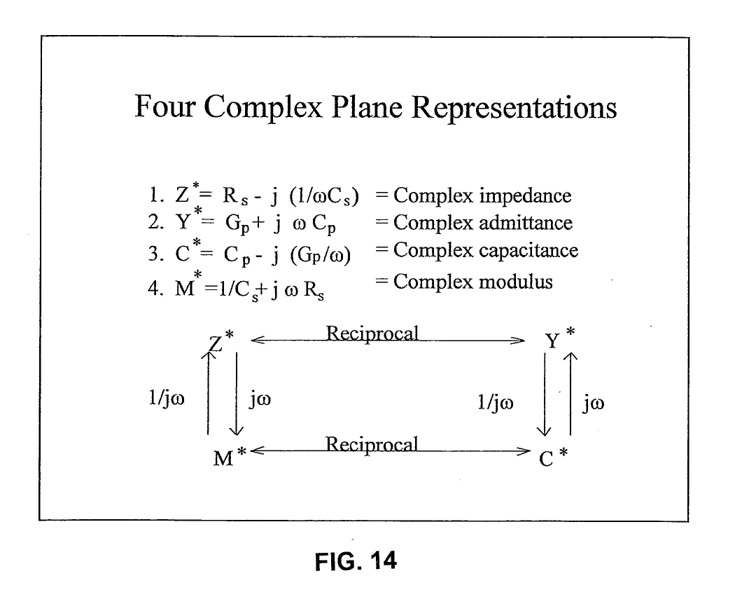

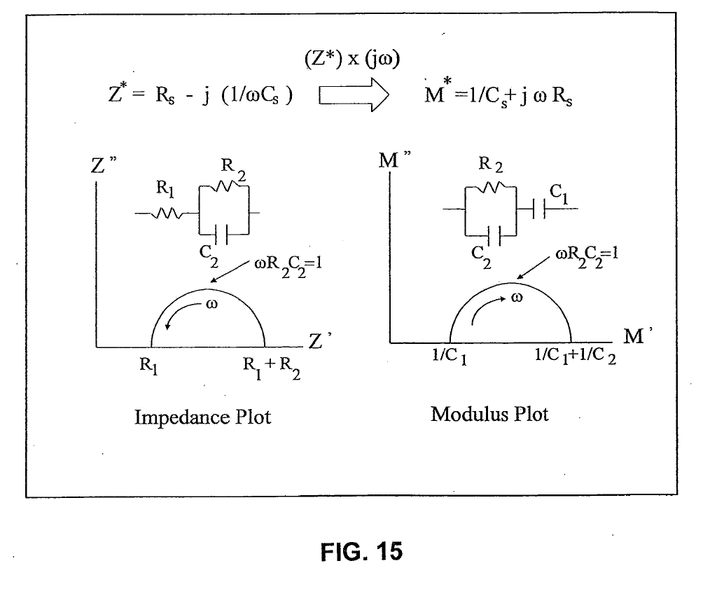

The biodiesel blend sample is tested and data is acquired by treating the sample as a series R—C combination. (See FIG. 13) The acquired sample data is converted by inversion of the weighting of the bulk media contribution to the total measured data response, wherein the value C₂ is typically a small value (See FIG. 14). This conversion minimizes the interfacial contribution of the bulk media, wherein the value C₁ is typically a large value (See FIG. 15). The real modulus transformation (M′) calculated for each biofuel sample is divided by the value (2*PI) in order to disguise the identity.

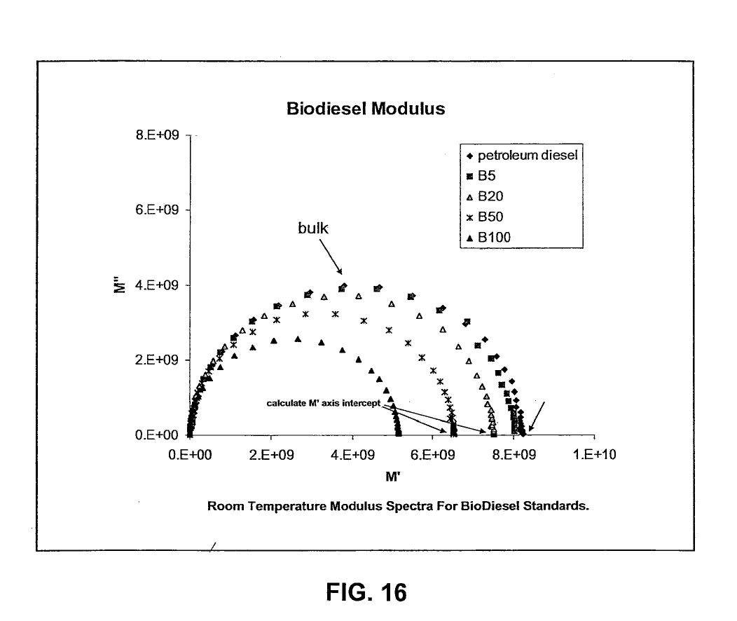

The biodiesel modulus spectra for the dedicated testing standards are provided in FIG. 16. The modulus data element M″ is plotted against the modulus data element M′. Data points for a petroleum diesel sample, as well as B5, B20, B50, and B100 were plotted. The complex impedance values (Z*) is converted to a complex modulus representation (M*) in order to inversely weight and isolate the bulk capacitance value from any interfacial polarization present within the sample. The M′ high frequency intercept via a semicircular fitting routine is then calculated.

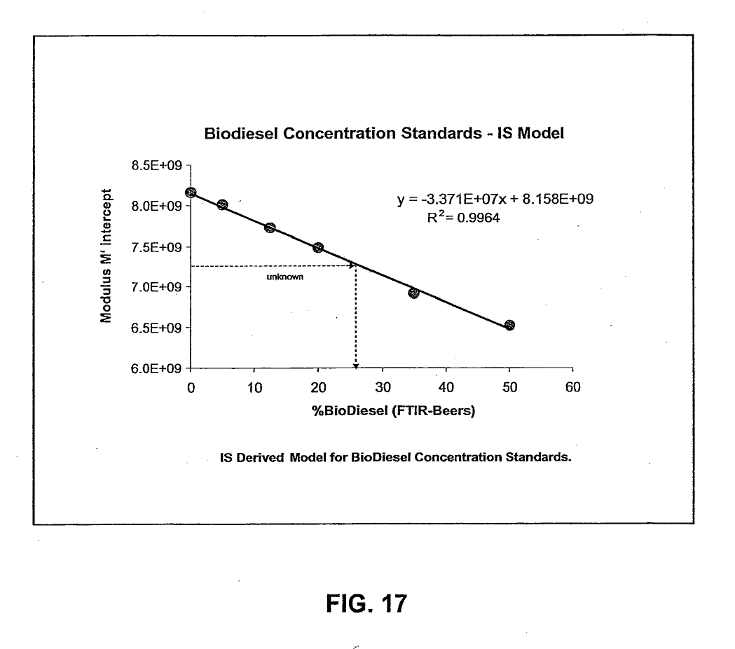

The biodiesel concentration standard, for which the impedance spectroscopy process will be measured against, is shown in FIG. 17. The previously calculated modulus (M′) intercept was plotted against the biodiesel concentration, as determined by the FTIR method. Equation Set 3 represents the derived algorithm.

y=−3.371E+07x+8.158E+09 Equation Set 3

where x=% biodiesel, and R²=0.9964

Biofuel samples are tested using the analyzer 12. The impedance data measurement is focused upon the biofuel sample while the electrode influence and probe fixturing are minimized.

In an alternative embodiment, fuel analyzer system 10 and methods of the present invention are used to determine the FAME concentration in heating fuel. The heating fuel sample is tested in a similar manner as that described for the biodiesel fuel blend. Alternatively, the system 10 can be used to analyze cutting fluids, engine coolants, heating oil (either petroleum diesel or biofuel) and hydrolysis of phosphate ester, which is used a hydraulic fluid (power transfer media).

In an alternative embodiment, the system 10 analyzes a biodiesel blend sample for the presence of substances selected from a group including second phase materials, fuel additives, glycerol, residual alcohol, moisture, unreacted feedstock (triacylglycerides), monoglycerides, diglycerides, and free (unreacted) fatty acids. In yet another alternative embodiment, the system 10 analyzes a biodiesel blend sample for the concentration of substances selected from a group including second phase materials, fuel additives, methanol, glycerol, residual alcohol, moisture, unreacted feedstock (triacylglycerides), monoglycerides, diglycerides, and free (unreacted) fatty acids.

It is specifically intended that the present invention not be limited to the embodiments and illustrations contained herein, but include modified forms of those embodiments including portions of the embodiments and combinations of elements of different embodiments.

The following United States patent documents are hereby incorporated by reference in their entirety herein. U.S. Pat. No. 6,278,281; U.S. Pat. No. 6,377,052; U.S. Pat. No. 6,380,746; U.S. Pat. No. 6,839,620; U.S. Pat. No. 6,844,745; U.S. Pat. No. 6,850,865; U.S. Pat. No. 6,989,680; U.S. Pat. No. 7,043,372; U.S. Pat. No. 7,049,831; U.S. Pat. No. 7,078,910; U.S. Patent Appl. No. 2005/0110503; and U.S. Patent Appl. No. 2006/0214671.

Although the invention has been described in detail with reference to preferred embodiments, variations and modifications exist within the scope and spirit of the invention as described and defined in the following claims.

1. An impedance spectroscopy (IS) system for characterizing a property of fuel, the system comprising appropriately coupled analysis means, a graphical user interface means (GUI), memory storage means and probe means and sample means:
the analysis means includes a logic controller, a modulus converter and an impedance converter, the logic controller, memory storage device, modulus converter and impedance converter being electronically coupled, the logic controller being configured to run a computer executable function and to receive and analyze data from the modulus converter and the impedance converter;
the GUI being coupled to the analysis means;
the memory storage means being coupled to the analysis means and optionally to the GUI, the memory storage device configured to receive and store data; and
the probe means being configured to interface with a fuel sample, and to transmit excitation voltage to a fuel sample at a plurality of frequencies, to receive fuel IS data from the fuel sample and to transmit the IS data to the logic controller;
wherein the logic controller characterizes the fuel at least in part using the IS data transmitted to the logic controller from the probe and a computer executable program adapted to determine fuel sample characteristics based in part upon the IS data.

2. A system according to claim 1, wherein the system is hand-held.

3. A system according to claim 1, wherein the fuel is diesel.

4. A system according to claim 3, wherein the biodiesel percent by volume of the fuel sample is determined.

5. A system according to claim 3, wherein the property of the fuel sample is the acid number.

6. A system according to claim 3, wherein the property of the fuel sample to be characterized is residual methanol.

7. A system according to claim 3, wherein the property of the fuel sample to be characterized is percent by volume glycerol.

8. A system according to claim 3, wherein the logic controller includes an oxidation analyzer.

9. A system for analyzing a fuel source comprising:
a probe for measuring the fuel source, the probe configured to transmit an excitation voltage into the fuel source and receive fuel source impedance spectroscopy (IS) data based at least in part upon the transmitted excitation voltage; and
an IS analysis device for analyzing IS data received by the probe, wherein the device determines the concentration of fatty acid alkyl esters within the fuel source based at least in part upon the IS data.

10. The system according to claim 9, wherein the fuel source includes biodiesel.

11. The system according to claim 9, wherein the probe is integral to a device having a combustion engine.

12. The system according to claim 9, wherein the IS analysis device further comprises a logic controller, modulus converter and impedance converter, the logic controller controls the modulus converter and impedance converter for retrieving, saving and analyzing IS data.

13. The system according to claim 10, wherein the fuel source concentration of fatty acid methyl ester (FAME) is determined, the FAME concentration is based at least in part upon the fuel source IS data.

14. The system according to claim 12, wherein the logic controller includes a set of IS data analyzers configured to analyze fuel source species selected from the group consisting of fuel blend concentration, water, glycerin, oxidation, fuel contaminants, alcohol, and acids.

15. An impedance spectroscopy (IS) system for determining biodiesel concentration of a biofuel source comprising:
an IS probe configured to transmit an excitation voltage to a fuel sample, to receive fuel source impedance spectroscopy (IS) data from the fuel sample, and to transmit IS data to a logic controller;
a logic controller configured to run a computer executable function, wherein the controller determines the concentration of fatty acid alkyl esters within the fuel sample based at least in part on the IS data and the computer executable function.

16. An impedance spectroscopic (IS) system for analyzing a biofuel sample comprising:
a probe configured to receive IS data when joined with a biofuel sample;
a logic controller configured to run a computer executable function, wherein the controller determines the concentration of fatty acid alkyl esters within the fuel sample based at least in part on IS data and the computer executable function,
wherein the IS data is based at least in part upon the response to an excitation voltage applied to the biofuel sample.

17. The system according to claim 16, wherein the fatty acid alkyl esters are fatty acid methyl esters.

18. The system according to claim 16, wherein the biofuel sample includes biodiesel.

19. The system according to claim 18, wherein the biofuel sample concentration of methanol is determined.

20. The system according to claim 18, wherein the system is handheld.

21. The system according to claim 16 wherein the system is in-line.

22. A system according to claim 16 wherein the system is deployed within a biofuel reservoir.
